(19) United States
(12) Patent Application Publication
(10) Pub. No.: US 2002/0102557 A1
(43) Pub. Date: Aug. 1, 2002

(54) Self -complementary molecular probe

(76) Inventors: Maria C. Gentile-Davey, Pleasant Prairie, WI (US); Clifford M. Chan, Grayslake, IL (US); Kristin Elmore, Waukegan, IL (US); James A. Rhoads, Mundelein, IL (US)

Correspondence Address: Steven F. Weinstock, ABBOTT LABORATORIES, D-377/AP6D, 100 Abbott Park Road, Abbott Park, IL, 60064-6050, US

(21) Appl. No.: 09/774,411

(22) Filed: Jan. 31, 2001

(51) Int. Cl. C12Q 1/68; C12P 19/34; C07H 21/04

(52) U.S. Cl. 435/6; 435/91.1; 536/24.3

(57) ABSTRACT

Disclosed herein is a molecular probe comprising a 3′ portion; a 5′ portion; and a center portion, that is complementary to a target sequence between the 3′ and 5′ portions. The 3′ portion is complementary to the 5′ portion such that when the 3′ portion is hybridized to the 5′ portion the probe has a stem loop structure. Additionally, parts of either or both of the 3′ or 5′ portions of the probe are complementary to the target sequence. Methods for using such probes are also provided.

### TECHNICAL FIELD OF THE INVENTION

The present invention relates to molecular biology, and more particularly, to nucleic acid reagents and detection of nucleic acids in a test sample.

### BACKGROUND OF THE INVENTION

Many processes currently are available for detecting a particular nucleic acid sequence (variously referred to as a “target sequence”) in a test sample. Such processes often times involve amplifying a target sequence to increase the number of target sequences, prior to detecting the target sequence. Amplification reactions such as the polymerase chain reaction (PCR), ligase chain reaction (LCR), gap ligase chain reaction (GLCR), strand displacement amplification (SDA), and target mediated amplification (TMA) have been previously described in the literature and are examples of various means available for amplifying a target sequence. (See, e.g., Roche Molecular Systems, Inc., Current Opinion in Biotechnology 4:41-47, 1993). Oligonucleotide probes are sometimes employed for detecting products from amplification reactions such as those mentioned above.

Homogeneous techniques for detecting target sequences also are currently available. Typically, homogeneous techniques for detecting target sequences involve a probe that has been modified such that it can be detected during the course of an amplification reaction. Hence, the reagents for the amplification and detection of a target sequence can be added to a single reaction vessel. A so-called “Taq-Man” assay is an example of homogeneous nucleic acid detection technique that employs a modified probe. In particular, TaqMan probes comprise a pair of labels. One of the labels is capable of emitting a detectable signal. However, the probe also is labeled with a quenching moiety, in close enough proximity to the emitting moiety, that prevents the detection of the signal emitted from the emitting label. During the course of, for example PCR, TaqMan probes hybridize to the target sequence while it is being amplified. The enzyme responsible for amplifying a target sequence also degrades any hybridized probe in its path. Accordingly, the labels on the probe are separated and the signal from the emitting label can be detected. As a result, the presence or absence of a target sequence in a test sample can be detected.

Another example of a probe that has been modified such that it can be used in a homogeneous amplification assay is a “molecular beacon”. Molecular beacons have been described in U.S. Pat. No. 5,925,517. Briefly, a molecular beacon is a single nucleic sequence that has self-complementary ends and a center region that hybridizes to a target sequence. At appropriate temperatures, the ends of a molecular beacon hybridize to form a stem loop structure wherein the self complementary ends form the stem of the stem loop structure and the center (target specific region) forms the loop of the stem loop structure. Additionally, molecular beacons typically are labeled at the ends with a quenching moiety and an emitting moiety. Hence, when a molecular beacon is in the stem loop configuration, the quenching moiety is close enough to the emitting moiety to prevent significant signal detection. However, in the presence of a target sequence, the affinity of the center region for the target overcomes the affinity that the ends of the beacon have for each other. As a result, the beacon hybridizes to the target sequence and holds the ends of the beacon apart. The signal from the emitting moiety is therefore not quenched by the quenching moiety. Consequently, a signal from a molecular beacon hybridized to its target can be detected and indicates the presence of the target sequence.

As mentioned above, molecular beacons contain nucleotides that do not participate in hybridizing to a target sequence. In particular, the ends of a molecular beacon are self-complementary and are not designed to hybridize to a target sequence. Hence, molecular beacons are longer than probes that completely hybridize to a target sequence, such as probes employed in TaqMan assays described above. Unfortunately, the increased length of a molecular beacon becomes a concern when assays requiring greater specificity are desired and longer target regions are therefore necessary. In particular, as these probes get longer, they begin to push beyond the boundaries of reliable nucleic acid synthesis using the available synthetic techniques. There is therefore a need for a more specific molecular beacon that reliably can be synthesized using existing techniques.

### BRIEF SUMMARY OF THE INVENTION

The present invention provides a molecular probe comprising a 3′ portion; a 5′ portion; and a center portion, that is complementary to a target sequence between the 3′ and 5′ portions. The 3′ portion is complementary to the 5′ portion such that when the 3′ portion is hybridized to the 5′ portion the probe has a stem loop structure. Additionally, parts of either or both of the 3′ or 5′ portions of the probe are complementary to the target sequence. As with other probes the probe provided herein can comprise nucleic acid or nucleic acid analogs. Hence, the center portion of the probe may be connected to the end portions with, for example, traditional phosphodiester or peptide bonds. Methods for using such probes are also provided.

### BRIEF DESCRIPTION OF THE DRAWINGS

FIGS. 1a and 1b schematically depicts a single stranded probe capable of forming a stem loop structure and the stem loop structure.

FIGS. 2a, 2b, and 2c schematically depict different stem loop probes.

### DETAILED DESCRIPTION OF THE INVENTION

The present invention provides a stem loop probe design that contains a target specific region while being capable of reliable synthesis using existing nucleic acid synthesis procedures. Probes according to the present invention form a stem loop structure in a manner similar to a molecular beacon or molecular switch described in U.S. Pat. Nos. 5,925,517 and 5,118,801 respectively (both of which are herein incorporated in their entirety by reference). However, unlike a molecular beacon, the target specific portions of probes disclosed herein extend into one or both arms of the stem loop structure. Such probe designs accordingly have greater specificity without sacrificing the ability to manufacture such structures.

As previously mentioned, probes according to the present invention are single stranded and comprise a 3′ portion and a 5′ portion which are complementary to each other. The 3′ and 5′ portions of the probes (or “end portions”) are separated by the center portion of the probe which forms a loop when the 3′ and 5′ portions are hybridized. The “3′ portion” and the “5′ portion” refer to the last several nucleotides at the 3′ and 5′ ends of the probe. The exact length of these portions is usually between 2 and 10 nucleotides long, more preferably between 3 and 8 nucleotides long, and most preferably between 4 and 7 nucleotides long. The exact length of these portions is largely a matter of choice based upon the melt temperature (“Tm”) of the center portion of the probe. In particular, the center portion of the probe should have a higher Tm than either the 3′ or 5′ portions so that, in the presence of a target sequence, the affinity that the center portion of the probe has for the target is stronger than the affinity that the 3′ and 5′ portions have for each other. As is known in the art, Tm is largely based upon the length and content of a sequence. For example, sequences having a higher G:C content have a higher melt temperature. Generally speaking, the number of nucleotides present in either of the end portions should be less than the number of nucleotides in the center portion of the probe.

Preferably, the end portions of a probe are completely complementary to each other. In other words, each nucleotide on one end of the probe finds its Watson-Crick binding partner on the other end of the probe in an alignment such that the corresponding nucleotides can hybridize to each other. However, mismatches are acceptable as long as the ends of the probe can hybridize to one another at ambient temperatures (i.e. 15° C. to 30° C.).

As previously mentioned, the probes taught herein comprise end portions and at least one of the end portions is complementary to the target sequence. Preferably, an entire end portion is complementary to the target sequence but it is not necessary for an entire end portion to be complementary to the target sequence. An end portion is considered complementary to the target if at least one, two three, four, or more of the nucleotides comprising the end portion are complementary to a given target sequence. Of course, both end portions of a probe may be complementary to the target in which case the target should contain self complementary sequences that are sufficiently spaced apart from each other to permit incorporation of a center portion into a probe for that target sequence.

In addition to at least one of the end portions, the center portion of probes described herein typically are complementary to a given target sequence. The length of the center portion of a probe is largely a matter of choice for those skilled in the art and largely based upon the target sequence. Typically, a center portion is between 8 and 50 nucleotides, more typically between 10 and 40 nucleotides, and most preferably between 15 and 30 nucleotides.

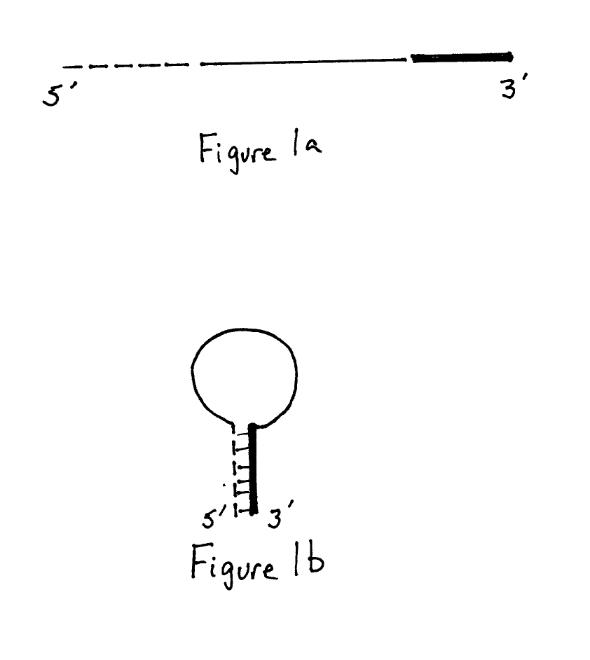

Turning to the Figures, FIG. 1a schematically depicts a single stranded probe having a 5′ portion (dashed line), a 3′ portion (bolded line) and a center portion (straight line). FIG. 1b demonstrates the probe in the stem loop configuration as a result of the 3′ and 5′ portions hybridizing to each other.

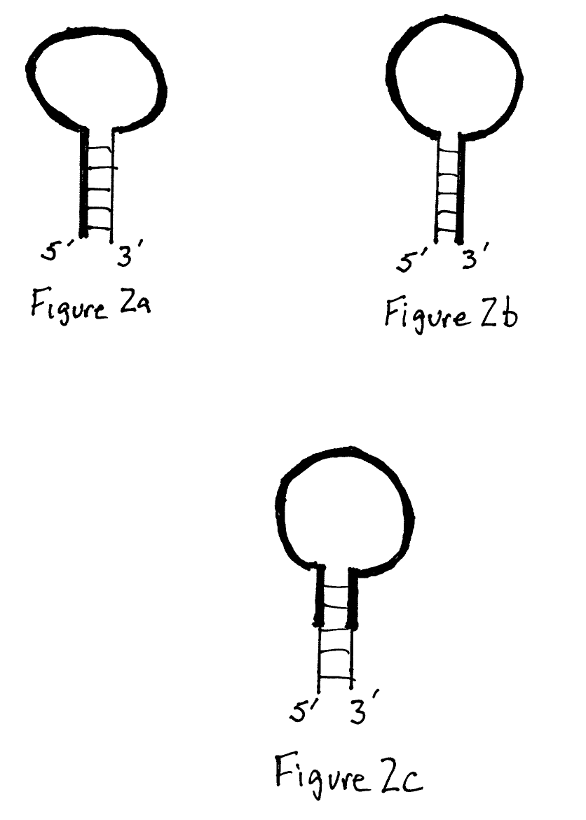

In addition to the center portion, probes according to the present invention, have (i) 5′ portions, or a part thereof, that hybridize to a target sequence, (ii) 3′ portions, or a part thereof, that hybridize to a target sequence; or (iii) 3′ and 5′ portions, or parts of both that hybridize to a target sequence. FIGS. 2a -2c schematically exemplify different embodiments of the invention. FIGS. 2a -2c depict stem loop structures where different portions of the stem loop structures are target specific or otherwise hybridize to a selected target sequence. The target specific regions of the structures shown in FIGS. 2a -2c are shown by bolded lines. For example, in FIG. 2a, the 5′ portion and the center portion (or loop) are specific for the target sequence; in FIG. 2b the 3′ portion and the center portion (or loop) are specific for the target sequence; and in FIG. 2c, the loop as well as parts of both the 3′ and 5′ portions are specific for the target sequence.

The probes described herein typically are nucleic acid sequences such as DNA or RNA and have a defined base sequence suitable for the desired target. Such sequences can be from natural or synthetic sources and can routinely be synthesized using a variety of techniques currently available. For example, a sequence of DNA can be synthesized using conventional nucleotide phosphoramidite chemistry and the instruments available from Perkin Elmer/Applied Biosystems, Div., (Foster City, Calif.). The probes may also incorporate nucleotide analogs such as uncharged nucleic acid analogs including but not limited to peptide nucleic acids (PNAs) which are disclosed in International Patent Application WO 92/20702 or morpholino analogs which are described in U.S. Pat. Nos. 5,185,444, 5,034,506, and 5,142,047 all of which are herein incorporated by reference. Hence, when the term “nucleotide” is used herein, it is intended to include nucleotides and/or nucleotide analogs such as, for example those previously mentioned.

Labels can be incorporated into the probes using labeling methodologies well known in the art such as described in U.S. Pat. Nos. 5,464,746; 5,424,414; and 4,948,882 all of which are herein incorporated by reference. The term “label” as used herein refers to a molecule or moiety having a property or characteristic which is capable of detection. A label can be directly detectable, as with, for example, radioisotopes, fluorophores, chemiluminophores, enzymes, colloidal particles, fluorescent microparticles, fluorescence resonance energy transfer (FRET) pairs, and the like. Alternatively, a label may be indirectly detectable, as with, for example, specific binding members. It will be understood that directly detectable labels may require additional components such as, for example, substrates, triggering reagents, light, and the like to enable detection of the label. When indirect labels are used for detection, they are typically used in combination with a conjugate that generally is a specific binding member attached to a directly detectable label. As used herein, specific binding member means a member of a binding pair, i.e., two different molecules where one of the molecules through, for example, chemical or physical means specifically binds to the other molecule. In addition to antigen and antibody specific binding pairs, other specific binding pairs include, but are not intended to be limited to, avidin and biotin; haptens and antibodies specific for haptens; complementary nucleotide sequences; and the like.

Preferably, the probes are labeled with an energy transfer pair of labels such as a FRET pair or collisional quenching pair such as Dabcyl and a fluorophore. In cases where energy transfer labels are employed, they are preferably located on the probes such that one label is on any part of one end portion and the other label is on any part of the other end portion (preferably on the last nucleotide on the 5′ and 3′ end portions). In this manner, the signal from the labels is quenched in the stem loop configuration and not quenched in the open configuration when the probe is hybridized to a target sequence. These same locations are preferred when indirectly detectable labels are employed so that binding pair members are not hindered from binding due to stearic hindrance caused by having the labels too close to one another. The exact location for placing labels on a probe is largely a matter of choice based upon the labels selected but optimum spacing can be achieved empirically by testing probes with various label locations, in assays with the various probes.

The probes can be employed in assays designed to detect a target sequence in a test sample. The term “target sequence” refers to a nucleic acid sequence that is detected or both amplified and detected. The term “test sample” as used herein means anything suspected of containing a target sequence. The test sample is or can be derived from any source, such as for example, biological sources including blood, plasma, ocular lens fluid, cerebral spinal fluid, milk, ascites fluid, synovial fluid, peritoneal fluid, amniotic fluid, tissue, fermentation broths, cell cultures, products of an amplification reaction, nucleic acid synthesis products and the like. Test samples can also be from, for example, environmental or forensic sources including sewage or cloth. The test sample can be used directly as obtained from the source or following a pre-treatment to modify the character of the sample. Thus, the test sample can be pre-treated prior to use by, for example, preparing plasma from blood, isolating cells from biological fluids, homogenizing tissue, disrupting cells or viral particles, preparing liquids from solid materials, diluting viscous fluids, filtering liquids, distilling liquids, concentrating liquids, inactivating interfering components, adding reagents, purifying nucleic acids, and the like.

According to one method when the target sequence is known to be in sufficient copy number for direct detection, a test sample is contacted with a probe, as described herein, such that a probe target sequence hybrid is formed. A signal is then detected to determine the presence of the target sequence in the test sample. Of course, it may be necessary to dissociate any double stranded target sequences before the hybrid is formed but this can be accomplished, as is well known in the art, with heat or chemical means such as sodium hydroxide.

In cases where amplification of the target sequence is deemed necessary, the method above may be preceded by an nucleic acid amplification step using any of the nucleic acid amplification reactions which are, by now, well known in the art. For example, amplification reactions that can be employed in accordance with methods provided herein include LCR described in European Patent Number 320 308 and its variations, such as gap LCR described in U.S. Pat. No. 5,792,607 (herein incorporated by reference), NASBA or similar reactions such as TMA described in U.S. Pat. No. 5,399, 491 (herein incorporated by reference),and preferably PCR which is described in U.S. Pat. Nos. 4,683,195 and 4,683,202 (both of which are herein incorporated by reference).

What is claimed is:

1. A single stranded nucleic acid probe comprising:
(a) a 3′ portion;
(b) a 5′ portion; and
(c) a center portion between the 3′ and 5′ portions; wherein
(i) the 3′ portion is complementary to the 5′ portion such that when the 3′ portion is hybridized to the 5′ portion the probe has a stem loop structure;
(ii) the center portion is complementary to a target sequence; and
(iii) the 3′ or 5′ portion of the probe is complementary to the target sequence.

2. The probe of claim 1 wherein the 3′ portion is complementary to the target sequence.

3. The probe of claim 1 wherein the 5′ portion is complementary to the target sequence.

4. The probe of claim 1 wherein both the 3′ and 5′ portions are complementary to the target sequence.

5. A method of detecting a target sequence in a test sample comprising the steps of:
a) contacting the test sample with the probe of claim 1 to form a hybrid between the probe and target sequence; and
b) detecting any signal from the hybrid as an indication of the presence of the target sequence in the test sample.

6. The method of claim 5 wherein the target sequence is amplified prior to step a.
